(19) United States
(12) Patent Application Publication
(10) Pub. No.: US 2018/0074491 A1
(43) Pub. Date: Mar. 15, 2018

(54) INFORMATION PROCESSING DEVICE, INFORMATION PROCESSING METHOD, AND INFORMATION PROCESSING PROGRAM OF MOBILE OBJECT

(71) Applicant: Kabushiki Kaisha Toshiba, Minato-ku (JP)

(72) Inventors: Toshiaki NAKASU, Chofu (JP); Tsukasa IKE, Shinagawa-ku (JP); Yasunobu YAMAUCHI, Aoba-ku (JP)

(73) Assignee: Kabushiki Kaisha Toshiba, Minato-ku (JP)

(21) Appl. No.: 15/426,478

(22) Filed: Feb. 7, 2017

(30) Foreign Application Priority Data
Sep. 13, 2016 (JP) 2016-178613

(51) Int. Cl. G05D 1/00; B60W 50/10; B60W 40/08; A61B 5/0488; G06F 1/16

(52) U.S. Cl. CPC G05D 1/0061; B60W 50/10; B60W 40/08; B60W 2040/0872; A61B 5/0488; G06F 1/163; G05D 1/0088

(57) ABSTRACT

According to one embodiment, an information processing device has processing circuitry and a memory. The processing circuitry is configured to acquire myoelectric potential of one part of a body of a user, determine whether the myoelectric potential is higher than reference myoelectric potential, switch autonomous driving to manual driving when the myoelectric potential is higher than the reference myoelectric potential, and output running control information to a mobile object after switching the autonomous driving to the manual driving. The memory is configured to store information that is required for processing that the processing circuitry executes.

### CROSS-REFERENCE TO RELATED APPLICATIONS

This application is based upon and claims the benefit of priority from the prior Japanese Patent Application No. 2016-178613, filed on Sep. 13, 2016; the entire contents of which are incorporated herein by reference.

### FIELD

Embodiments described herein relate generally to an information processing device, an information processing method, and an information processing program of a mobile object.

### BACKGROUND

There has been proposed a mobile object, such as an automobile, which moves under autonomous driving when an occupant sitting inside does not perform manual driving.

However, even when the mobile object is moving under autonomous driving, an occupant would rather drive the mobile object manually as the need arises. Such being the case, autonomous driving can be switched to manual driving in some types of mobile object.

Using a hand-operated selector switch to switch autonomous driving to manual driving, however, poses a problem that a switching operation is troublesome.

An object of embodiments described herein is to provide an information processing device, an information processing method, and an information processing program of a mobile object, each of which is capable of readily switching autonomous driving to manual driving.

### BRIEF DESCRIPTION OF THE DRAWINGS

FIG. 1 is a block diagram of a mobile object of one embodiment;

FIG. 2 is a flowchart of processing circuitry;

FIG. 3 is a view of a user operating a steering wheel;

FIG. 4 is a view of a closed hand;

FIG. 5 is a view of an opened hand;

FIG. 6 is a view used to describe upward and downward tilts when the user wears a motion sensor on an arm;

FIG. 7 is a view used to describe rightward and leftward tilts when the user wears the motion sensor on the arm;

FIG. 8 is a view used to describe clockwise and counterclockwise tilts when the user wears the motion sensor on the arm; and

FIG. 9 is a view used to describe a hand gesture for a stop operation.

### DETAILED DESCRIPTION

According to embodiments, an information processing device of a mobile object movable under autonomous driving and manual driving by a user has processing circuitry and a memory. The processing circuitry is configured to acquire myoelectric potential of at least one part of a body of the user, determine whether the myoelectric potential is higher than reference myoelectric potential, switch the autonomous driving to the manual driving when the myoelectric potential is higher than the reference myoelectric potential, and output running control information to the mobile object after switching the autonomous driving to the manual driving. The memory is configured to store information that is required for processing that the processing circuitry executes.

Hereinafter, an information processing device 2 of a mobile object 1 according to one embodiment will be described. In this embodiment, the mobile object 1, such as an automobile, is switched from autonomous driving to manual driving according to information acquired from a gesture of an arm or a hand of a user. Normally, the user does not hold a steering wheel during autonomous driving. However, when the mobile object 1 is highly likely to collide with a wall or another vehicle, the user has to instantly switch autonomous driving to manual driving which enables a driving operation by the user. Hence, this embodiment will describe the information processing device 2 which enables the user to switch autonomous driving to manually driving when a need arises during autonomous driving.

### (1) Configuration of Mobile Object 1

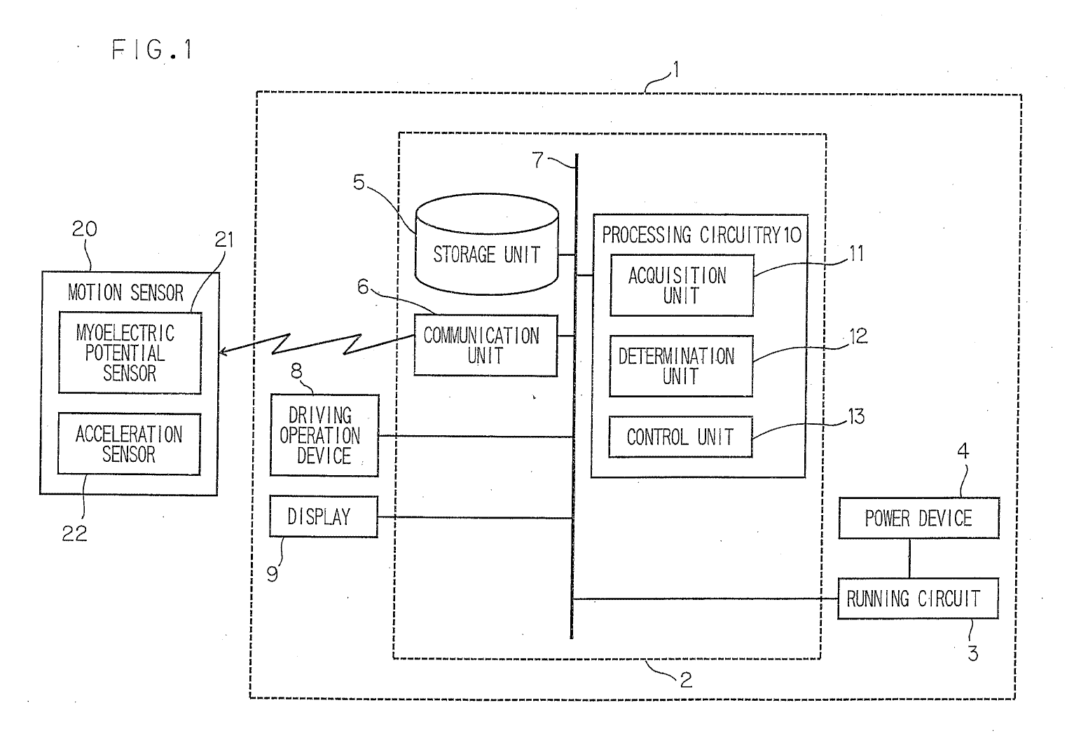

A configuration of the mobile object 1 will be described with reference to a block diagram of FIG. 1. The mobile object 1 is an automobile for an occupant (hereinafter, referred to as the user) to get in. The mobile object 1 is movable under manual driving by which the mobile object 1 runs according to a driving operation by the user and autonomous driving by which the mobile object 1 runs autonomously without the user having to perform a driving operation. The mobile object 1 includes the information processing device 2, a running circuit 3, a power device 4, a driving operation device 8, a display 9, and a motion sensor 20 having a myoelectric potential sensor 21 and an acceleration sensor 22.

The information processing device 2 is, for example, a dedicated or general purpose computer. Herein, a case where the information processing device 2 is equipped to the mobile object 1 will be described. It should be appreciated, however, that the information processing device 2 is not limited to a configuration described below and, for example, processing by the information processing device 2 may be executed on a cloud resource. The information processing device 2 includes processing circuitry 10, a storage circuit 5, a communication unit 6, and a bus 7 interconnecting the respective components. Respective processing functions performed by the information processing device 2 are preliminarily stored in the storage circuit 5 in the form of computer-executable programs.

The processing circuitry 10 includes an acquisition unit 11, a determination unit 12, and a control unit 13. FIG. 1 chiefly shows functions furnished in this embodiment by way of example. It should be appreciated, however, that functions furnished to the processing circuitry 10 are not limited to the functions shown in FIG. 1. Respective processing functions will be described below. The processing circuitry 10 is a processor that realizes a function corresponding to each program by reading out the program from the storage circuit 5 and executing the read program. The above has described a case with reference to FIG. 1 where the processing circuitry solely realizes processing functions furnished to the acquisition unit 11, the determination unit 12, and the control unit 13. However, the processing circuitry 10 may be formed by combining multiple independent processors to let each processor realize a furnished function by executing a corresponding program. Further, each processing function may be provided in the form of a program and the processing circuitry 10 may solely execute all programs. Furthermore, a particular function may be furnished to a dedicated, independent program execution circuit.

The term, “processor”, referred to above means circuitry represented by, for example, a CPU (Central Processing Unit), a GPU (Graphical Processing Unit), an ASIC (Application Specific Integrated Circuit), an SPLD (Simple Programmable Logic Device), a CPLD (Complex Programmable Logic Device), or an FPGA (Field Programmable Gate Array). The processor realizes a function by reading out and executing a corresponding program saved in the storage circuit 5. A program may be directly installed to an internal circuit of the processor instead of saving a program in the storage circuit 5. In such a case, the processor realizes a function by reading out and executing a corresponding program installed to the internal circuit.

The storage circuit 5 stores data or the like involved in various processing functions performed by the processing circuitry 10 as needed. The storage circuit 5 stores programs and other types of data. Examples of the storage circuit 5 include but not limited to a semiconductor memory element, such as a RAM (Random Access Memory) and a flash memory, a hard disk, and an optical disk. Processing executed by an internal storage circuit of the processing circuitry 10 may be alternatively executed by an external storage device of the information processing device 2. The storage circuit 5 may be a storage medium in which a program transferred via a LAN (Local Area Network), the Internet, or the like is downloaded and stored or transiently stored. The number of storage medium is not limited to one. Processing in this embodiment may be executed using a plurality of storage media. The storage media can adopt any one of the above configurations.

The motion sensor 20 is of a wristband type to be worn on a part of a body of the user and has the myoelectric potential sensor 21 and the acceleration sensor 22. The user may wear the motion sensor 20 on, for example, an arm. However, the motion sensor 20 is not necessarily worn on an arm and may be worn on any other appropriate part of the body, such as a wrist, an upper arm, a finger, a head, a thigh, and an ankle.

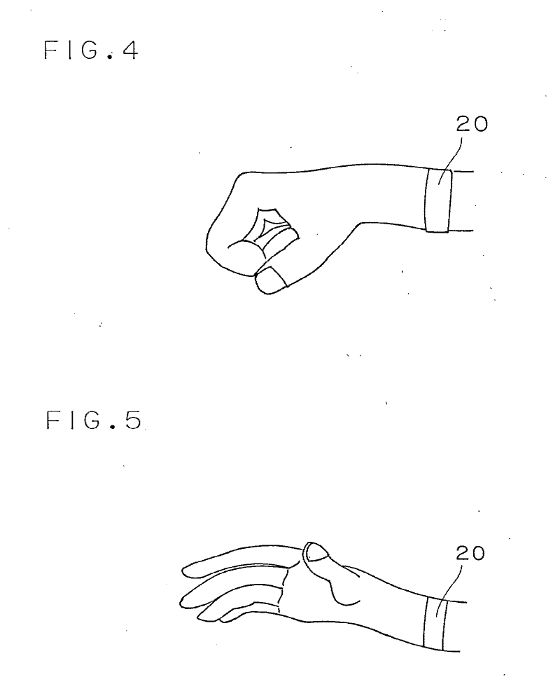

The myoelectric potential sensor 21 is provided with three sets of electrodes along an inner periphery of the wrist band and closely attached to an arm of the user. Each set of electrodes detects myoelectric potential in time sequence. When the user closes his hand as is shown in FIG. 4, amplitude of a waveform of myoelectric potential K increases. On the contrary, amplitude degreases when the user relaxes handgrip by opening his hand as is shown in FIG. 5. The myoelectric potential sensor 21 outputs, for example, an average value of the three sets of electrodes as the myoelectric potential K.

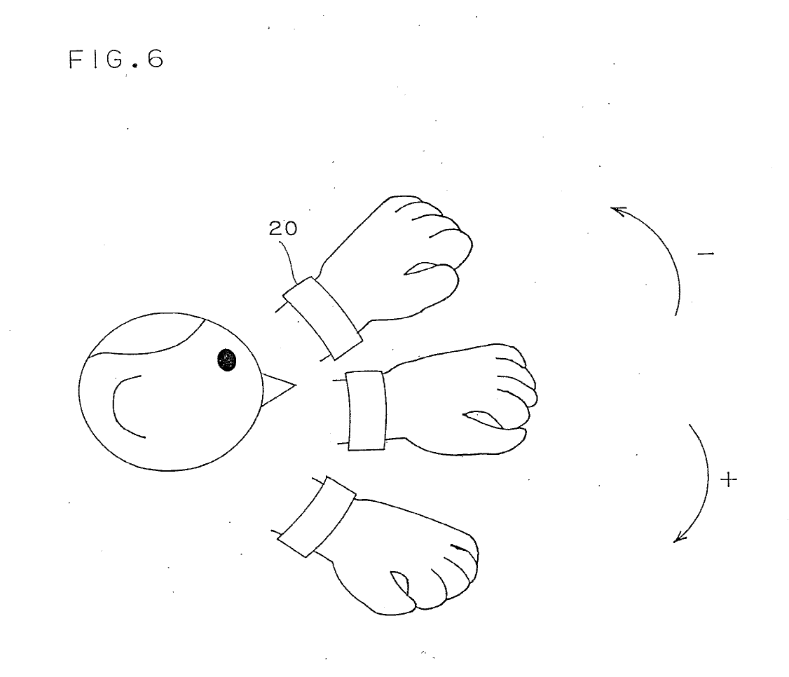

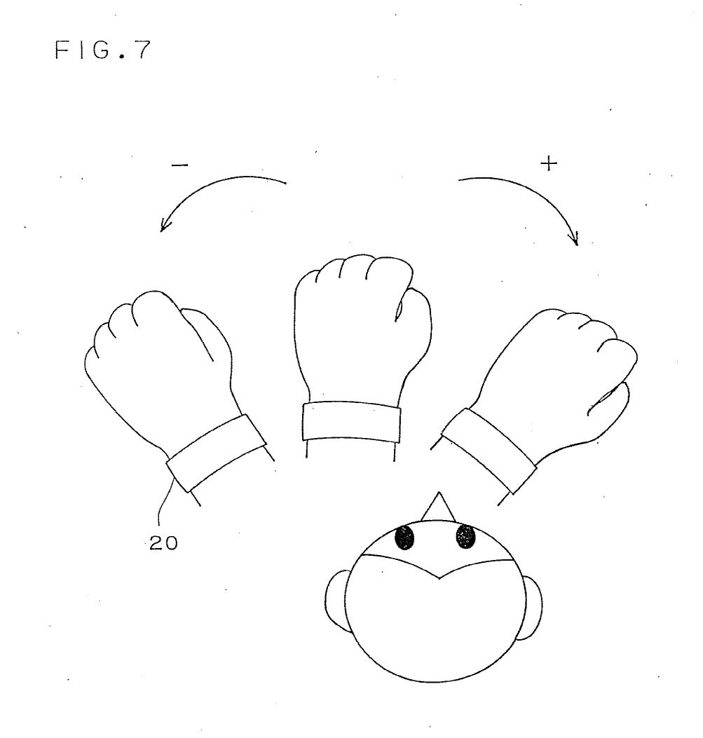

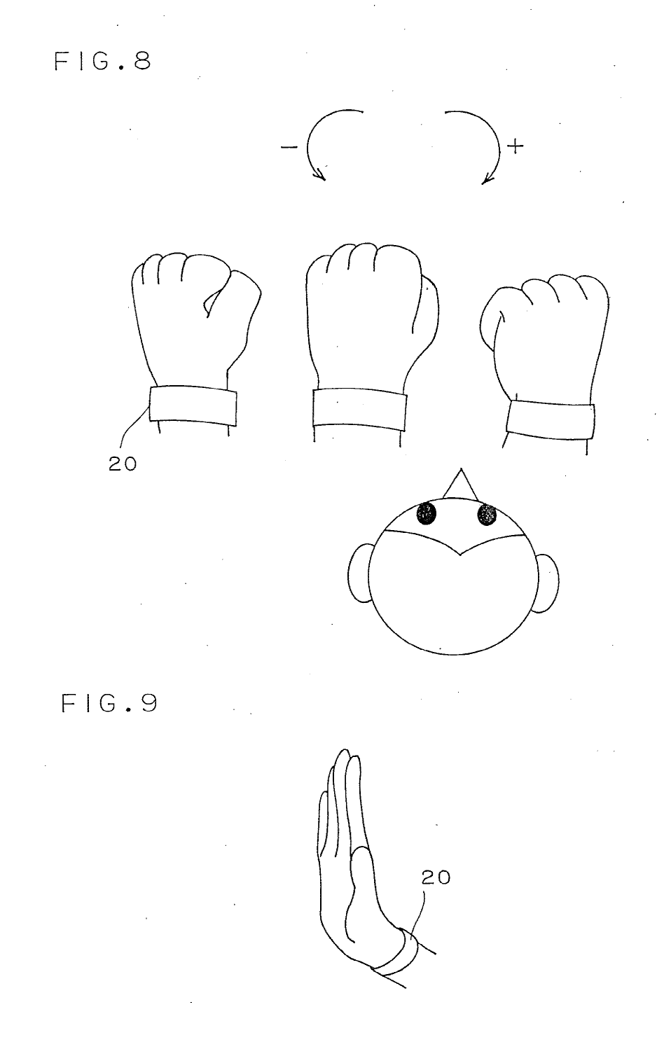

The acceleration sensor 22 detects a feature amount in a part of the body of the user. A feature amount is “a three-dimensional tilt θ” found from acceleration of three axes on a three-dimensional space detected by the acceleration sensor 22. The term, “three-dimensional tilt θ”, referred to herein is expressed by, for example, a pitch which is, as is shown in FIG. 6, a rotational angle in a top-bottom direction with respect to an axis pointing in a right-left direction when viewed from the acceleration sensor 22, a yaw which is, as is shown in FIG. 7, a rotational angle in the right-left direction with respect to an axis pointing in the top-bottom direction, and a roll which is, as is shown in FIG. 8, a rotational angle with respect to an axis pointing in a front-rear direction. These three parameters vary with a wearing position and a wearing angle of the motion sensor 20. In a case where the user wears the motion sensor 20 on an arm, as are shown in FIGS. 6 through 8, the three parameters are calculated to be rotational angles in the top-bottom direction, the right-left direction, and the clockwise or counterclockwise direction when viewed from the user. For example, let a tilt at a particular instant be a reference and a downward direction, a rightward direction, and a clockwise direction be positive. Then, a three-dimensional tilt θ is calculated as a relative tilt (rotational angle) with respect to the reference (for example, a tilt at an instant when an application starts). Alternatively, a trajectory may be calculated from acceleration alone. The pitch and the roll may be calculated on the assumption that an integrated vector of three-dimensional acceleration obtained from the acceleration sensor 21 is a gravitational acceleration direction.

The communication unit 6 is an interface which inputs information from and outputs information to the operation sensor 20 and an external device connected either by wire or radio. The communication unit 6 may be connected to a network to make communications. For example, the communication unit 6 acquires information on a location of a subject vehicle and also information on road conditions (accident, jamming, and so on) specified by a GPS.

The driving operation device 8 accepts various instructions and inputs of information from the user. The driving operation device 8 includes, for example, a steering wheel, an accelerator pedal, a brake pedal, and a direction indictor.

The display 9 displays various types of information on the mobile object 1. The display 9 is, for example, included in a car navigation system formed of a display device, such as a liquid crystal display, and displays a map image or the like.

The running circuit 3 controls the power device 4 including unillustrated motor, wheels, and so on, to be more specific, controls directions of the wheels, an engine, the motor, and so on for the mobile object 1 to move according to the running control information from the processing circuitry 10.

### (2) Configuration of Information Processing Device 2

A configuration of the information processing device 2 will be described with reference to the block diagram of FIG. 1. The processing circuitry 10 of the information processing device 2 includes the acquisition unit 11, the determination unit 12, and the control unit 13. The term, “autonomous driving”, referred to herein means that the mobile object 1 operates autonomously to take a right turn, a left turn, accelerate, decelerate, and stop. The term, “manual driving”, referred to herein means that the user himself holds and operates the steering wheel to drive the mobile object 1 while operating the accelerator pedal and the brake pedal. There are two critical points when autonomous driving is switched to manual driving.

A first point is in which manner an intention of the user to switch autonomous driving to manual driving is determined. In this embodiment, myoelectric potential K of an arm of the user is measured by the myoelectric potential sensor 21 and an intention to switch autonomous driving to manual driving is determined on the basis of the measured myoelectric potential K.

A second point is in which manner the mobile object 1 is run when autonomous driving is switched to manual driving.

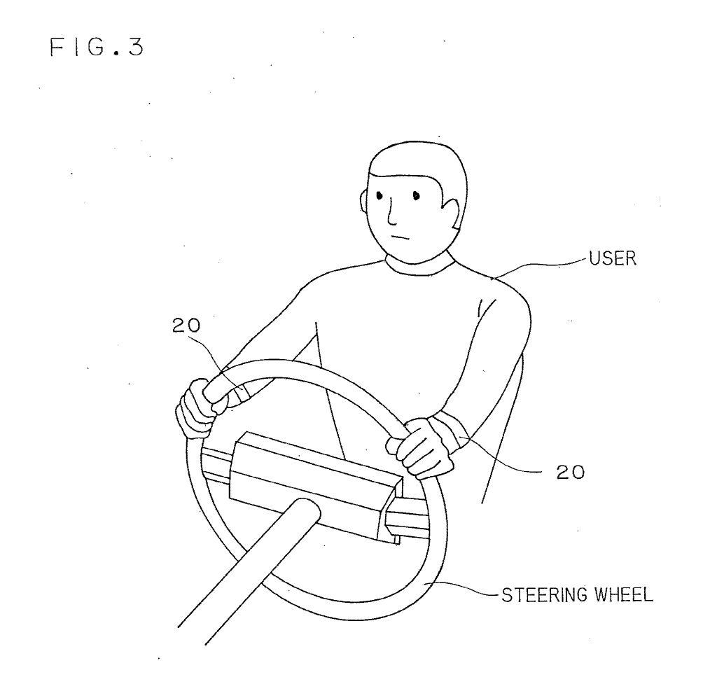

In this embodiment, when autonomous driving is switched to manual driving, the mobile object 1 is run by using a three-dimensional tilt θ of the arm of the user detected by the acceleration sensor 22. Also, in this embodiment, as is shown in FIG. 3, an operation target controlled by using a three-dimensional tilt θ of the arm is the steering wheel. The user may hold the steering wheel or may make a gesture to hold the steering wheel instead of actually holding the steering wheel.

The acquisition unit 11 acquires information from the motion sensor 20, more specifically, myoelectric potential K of the arm of the user from the myoelectric potential sensor 21 and a three-dimensional tilt θ of the arm of the user from the acceleration sensor 22 in time sequence. In this embodiment, the acquisition unit 11 is connected to the motion sensor 20 by radio via the communication unit 6.

The determination unit 12 determines whether the user is going to switch autonomous driving to manual driving in response to myoelectric potential K, to be more specific, on the basis of amplitude of myoelectric potential K. For example, the determination unit 12 determines that the user is going to switch autonomous driving to manual driving when myoelectric potential K is higher than reference myoelectric potential d0 The determination unit 12 occasionally determines whether the user is going to switch autonomous driving to manual driving by using a three-dimensional tilt θ in addition to myoelectric potential K.

The control unit 13 generates running control information indicating a manner in which to operate the steering wheel, according to a three-dimensional tilt θ of the arm and outputs the generated information to the running circuit 3. The control unit 13 may identify an operation of the steering wheel in response to a three-dimensional tilt θ of the arm by means of existing machine learning.

In the following, multiple embodiments will be described one by one as to a control method of switching the mobile object 1 from autonomous driving to manual driving by the user using the information processing device 2 configured as above.

### (3) First Control Method of Information Processing Device 2

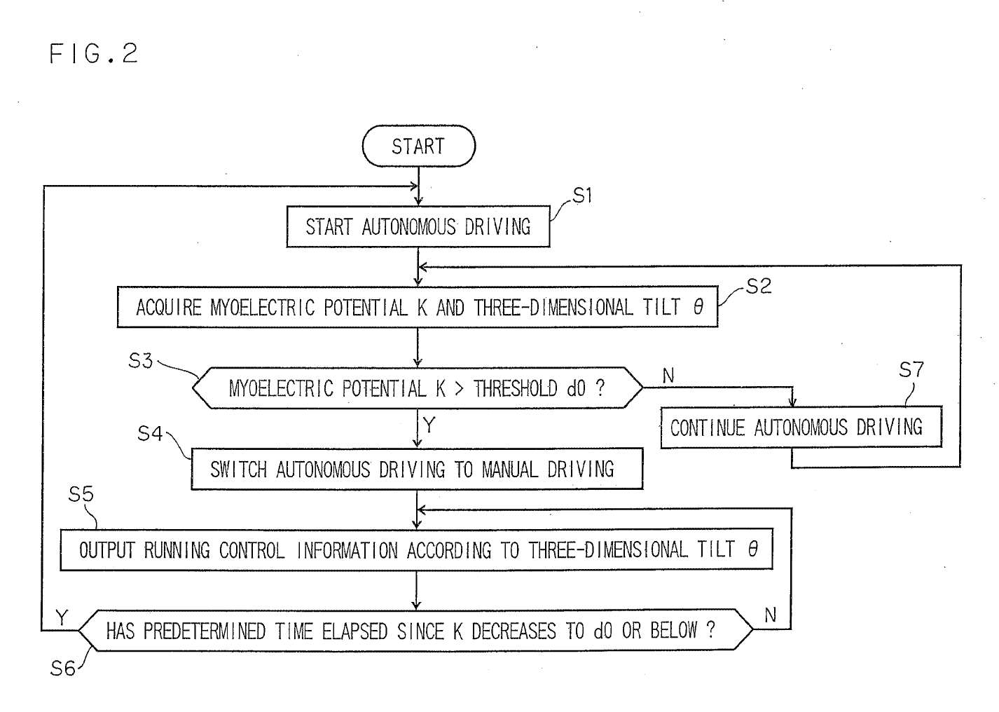

A first control method of the information processing device 2 will be described with reference to a flowchart of FIG. 2. The first control method switches autonomous driving to manual driving when myoelectric potential K is higher than the reference myoelectric potential d0.

In Step S1, the processing circuitry 10 starts autonomous driving. Subsequently, advancement is made to Step S2.

In Step S2, the acquisition unit 11 acquires myoelectric potential K and a three-dimensional tilt θ in time sequence.

In Step S3, the determination unit 12 determines whether a condition that myoelectric potential K is higher than the reference myoelectric potential d0 (K>d0) is satisfied. When K>d0, advancement is made to Step S4 (the case of Y). When K≦d0, advancement is made to Step S7 (the case of N).

In Step S4, given K>d0, the determination unit 12 switches autonomous driving to manual driving. Subsequently, advancement is made to Step S5.

In Step S5, because autonomous driving is switched to manual driving, the control unit 13 generates running control information indicating a manner in which to operate the steering wheel, according to a three-dimensional tilt θ and outputs the generated information to the running circuit 3. Subsequently, advancement is made to Step S6.

In Step S6, when a predetermined time has elapsed since myoelectric potential K decreases to or below the reference myoelectric potential d0, the determination unit 12 determines that the user no longer intends to operate the steering wheel, in which case the flow returns to Step S1 (the case of Y). When myoelectric potential K is higher than the reference myoelectric potential d0 or when the predetermined time has not elapsed since myoelectric potential K decreases to or below the reference myoelectric potential d0, the flow returns to Step S5 (the case of N).

In Step S7, given K≦d0, the determination unit 12 determines that the user has no intention to operate the steering wheel and continues autonomous driving, in which case the flow returns to Step S2.

### (4) Second Control Method

A second control method of the information processing device 2 uses thresholds d0 and d1 through dk in multiple steps when the determination unit 12 switches autonomous driving to manual driving on the basis of myoelectric potential K. In this embodiment, a description will be given to a case using thresholds (the reference myoelectric potential) d0 and d1 in two steps, where d1>d0.

When K>d1, the determination unit 12 switches autonomous driving to manual driving. Upon switching to manual driving, the control unit 13 outputs running control information indicating a manner in which to operate the steering wheel, according to a three-dimensional tilt θ to the running circuit 3.

When d1≧K>d0, the determination unit 12 determines that the control unit 13 controls driving of the mobile object 1 according to a weighted sum of manual driving and autonomous driving. More specifically, a switching degree to manual driving is increased as myoelectric potential K become higher. For example, in a case where the user operates the steering wheel to travel straight ahead while the mobile object 1 is being turned to the right by 90° by autonomous driving, the mobile object 1 is turned by an angle closer to a straight-ahead direction as myoelectric potential K becomes higher. That is to say, the control unit 13 turns the mobile object 1 according to a weighted sum of a straight-ahead angle of 0° by manual driving and a turning angle of 90° by autonomous driving. Gripping strength and a switching degree to manual driving may not necessarily correspond linearly and may correspond non-linearly like a sigmoid function.

When K≦d0, the determination unit 12 continues autonomous driving.

### (5) Third Control Method

A third control method of the information processing device 2 varies a time taken to switch autonomous driving to manual driving according to myoelectric potential K.

When myoelectric potential K is higher than a myoelectric potential threshold e (K>e), where e>d0, the determination unit 12 immediately switches autonomous driving to manual driving. Upon switching to manual driving, the control unit 13 outputs running control information indicating a manner in which to operate the steering wheel, according to a three-dimensional tilt θ to the running circuit 3.

When d0<K≦e, the determination unit 12 switches autonomous driving to manual driving after a predetermined time (for example, ten seconds). Upon switching to manual driving, the control unit 13 outputs running control information indicating a manner in which to operate the steering wheel, according to a three-dimensional tilt θ to the running circuit 3.

### (6) Fourth Control Method

A fourth control method of the information processing device 2 switches autonomous driving to manual driving when a three-dimensional tilt θ of the arm exceeds a first angle threshold α after myoelectric potential K rises above the reference myoelectric potential d0. In particular, by allocating an extremely rare motion in normal situations, the user is allowed to make a manual operation only when the user intends to.

When a three-dimensional tilt θ of the arm detected by the acceleration sensor 22 exceeds the first angle threshold a after myoelectric potential K rises above the reference myoelectric potential d0 (K>d0), the determination unit 12 switches autonomous driving to manual driving. Upon switching to manual driving, the control unit 13 outputs running control information indicating a manner in which to operate the steering wheel, according to a three-dimensional tilt θ to the running circuit 3.

When 8 a even after myoelectric potential K rises above the reference electric potential d0 (K>d0), the determination unit 12 continues autonomous driving.

For example, when the user moves the arm noticeably after myoelectric potential K rises above the reference myoelectric potential d0, autonomous driving is switched to manual driving.

In the fourth control method, too, the first angle threshold a may be provided in multiple steps to increase a degree of manual driving with respect to autonomous driving as a three-dimensional tilt θ of the arm becomes higher after myoelectric potential K rises above the reference myoelectric potential d0 (K>d0).

### (7) Fifth Control Method

A fifth control method of the information processing device 2 varies a switching speed from autonomous driving to manual driving according to magnitude of a three-dimensional tilt θ of the arm after myoelectric potential K rises above the reference myoelectric potential d0.

When a three-dimensional tilt θ after myoelectric potential K rises above the reference myoelectric potential d0 (K>d0) is greater than a second angle threshold β (θ>β), the determination unit 12 immediately switches autonomous driving to manual driving. Upon switching to manual driving, the control unit 13 outputs running control information indicating a manner in which to operate the steering wheel, according to a three-dimensional tilt θ to the running circuit 3.

When a three-dimensional tile θ after myoelectric potential K rises above the reference myoelectric potential d0 (K>d0) is equal to or less than the second angle threshold β (θ≦β), the determination unit 12 switches autonomous driving to manual driving after a predetermined time (for example, ten seconds). Upon switching to manual driving, the control unit 13 outputs running control information indicating a manner in which to operate the steering wheel, according to a three-dimensional tilt θ to the running circuit 3.

### (8) Sixth Control Method

A sixth control method of the information processing device 2 varies a switching speed from autonomous driving to manual driving with a rapidity of change, V, with which myoelectric potential K rises above the reference myoelectric potential d0. For example, when the user holds the steering wheel quickly, autonomous driving is quickly switched to manual driving.

When a rapidity of change, V, of myoelectric potential K is higher than a rapidity threshold v0 (V>vo), the determination unit 12 immediately switches autonomous driving to manual driving. Upon switching to manual driving, the control unit 13 outputs running control information indicating a manner in which to operate the steering wheel, according to a three-dimensional tilt θ to the running circuit 3.

When V≦v0, the determination unit 12 switches autonomous driving to manual driving after a predetermined time (for example, ten seconds). Upon switching to manual driving, the control unit 13 outputs running control information indicating a manner in which to operate the steering wheel, according to a three-dimensional tilt θ to the running circuit 3.

Even in the sixth control method, too, the rapidity threshold v0 may be provided in multiple steps to gradually make a switching time shorter or longer (for switching to take place faster or slower).

### (9) Seventh Control Method

A seventh control method of the information processing device 2 switches autonomous driving to manual driving when myoelectric potential K remains above the reference myoelectric potential d0 for a certain time. When a hand gripping state at a strength above a certain level continues for a predetermined time or longer, the determination unit 12 determines that the user intends to operate the steering wheel. For example, when the user keeps holding the steering wheel for a considerable time, autonomous driving is switched to manual driving. Hence, even in an occasion where a force is exerted transiently when the user does not intend to operate the steering wheel, an erroneous operation can be prevented.

When K>d0 and a time over which the user keeps holding the steering wheel (hereinafter, referred to as a duration time) t is longer than a time threshold T0 (t>T0), the determination unit 12 switches autonomous driving to manual driving.

When t≦T0, the determination unit 12 continues autonomous driving.

Even in the seventh control method, too, the time threshold T0 may be provided in multiple steps to perform a control according to a weighted sum of manual driving and autonomous driving in each step. In such a case, a degree of autonomous driving is increased as the duration time t becomes longer.

### (10) Eighth Control Method

An eighth control method of the information processing device 2 switches autonomous driving to manual driving when myoelectric potential K is higher than the reference myoelectric potential d0 according to relevance of angular variations between both arms of the user. For example, when the user wears the motion sensor 20 on the both arms, relevance of angular variations between the both arms of the user increases for a gesture to turn the steering wheel by holding the steering wheel with both hands. Hence, relevance S of angular variations is found from three-dimensional tilts θ of the both arms calculated by the respective motion sensors 20 worn on the both arms, and whether to switch autonomous driving to manual driving is determined according to the relevance S thus found.

According to “relevance S”, a three-dimensional tilt θ of the left hand and a three-dimensional tilt θ of the right hand are related to each other when both are within a reference range for a predetermined time, and relevance S increases as a difference of the three-dimensional tilts θ between the both hands becomes smaller.

When myoelectric potential K is higher than the threshold d0 (K>d0) and relevance S of angular variations between the both arms is higher than a first relevance threshold s0 (S>s0), the determination unit 12 switches autonomous driving to manual driving.

When S≦s0, the determination unit 12 continues autonomous driving.

Even in the eighth control method, too, the first relevance threshold s0 may be provided in multiple steps to increase a degree of manual driving as relevance S increases. In such a case, a running control is performed according to a weighted sum of manual driving and autonomous driving.

### (11) Ninth Control Method

A ninth control method of the information processing device 2 increases a speed with which to switch autonomous driving to manual driving according to relevance S of angular variations between the both arms when myoelectric potential K is higher than the reference myoelectric potential d0.

When myoelectric potential K is higher than the reference myoelectric potential d0 (K>d0) and relevance S of angular variations between the both arms is greater than a second relevance threshold m (S>m), the determination unit 12 immediately switches autonomous driving to manual driving.

When S≦m, the determination unit 12 switches autonomous driving to manual driving after a predetermined time (for example, ten seconds).

Even in the ninth control method, too, the second relevance threshold m may be provided in multiple steps to increase the switching speed as relevance S increases.

### [Modification]

A modification will now be described. In the embodiments described above, an operation target is the steering wheel. However, the operation target may be, for example, a brake pedal or an accelerator pedal of the mobile object 1 instead. In a case where the operation target is the brake pedal, the determination unit 12 may switch autonomous driving to manual driving when myoelectric potential K rises above the threshold d0 as the user moves his hand forward with the palm facing front as is shown in FIG. 9. The control unit 13 acquires a three-dimensional tilt θ of the arm when the hand is moved forward with the palm facing front and immediately puts a brake.

The acceleration sensor 22 may calculate a tilt θ by using an angular velocity or geomagnetism besides acceleration. For example, a tilt θ may be calculated by a nine-axes sensor capable of acquiring three-dimensional acceleration, angular velocity, and geomagnetism in a part where the motion sensor 20 is worn. For example, a tilt of a sensor calculated from values of the three-dimensional acceleration, angular velocity, and magnetism by using a technique disclosed in Non-Patent Literature 1 may be deemed as a three-dimensional tilt θ of a part of the user.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An information processing device of a mobile object movable under autonomous driving and manual driving by a user, the device comprising:
processing circuitry configured to:
acquire myoelectric potential of at least one part of a body of the user,
determine whether the myoelectric potential is higher than reference myoelectric potential,
switch the autonomous driving to the manual driving when the myoelectric potential is higher than the reference myoelectric potential, and
output running control information to the mobile object after switching the autonomous driving to the manual driving; and
a memory configured to store information that is required for processing that the processing circuitry executes.

2. The device according to claim 1, wherein the processing circuitry is configured to:
acquire at least acceleration in time sequence as to motion of the part of the body of the user; and
generate the running control information according to the acceleration after switching the autonomous driving to the manual driving.

3. The device according to claim 2, wherein the processing circuitry is configured to:
acquire a three-dimensional tilt in time sequence found from the acceleration of three axes on a three-dimensional space; and
generate the running control information according to the three-dimensional tilt after switching the autonomous driving to the manual driving.

4. The device according to claim 1, wherein the processing circuitry is configured to:
switch the autonomous driving to the manual driving when the myoelectric potential is higher than both of the reference myoelectric potential and a myoelectric potential threshold; and
switch the autonomous driving to the manual driving after a predetermined time when the myoelectric potential is higher than the reference myoelectric potential and equal to or lower than the myoelectric potential threshold.

5. The device according to claim 3, wherein the processing circuitry is configured to:
switch the autonomous driving to the manual driving when one of the acceleration and the three-dimensional tilt becomes greater than a first angle threshold after the myoelectric potential rises above the reference myoelectric potential.

6. The device according to claim 3, wherein the processing circuitry is configured to:
switch the autonomous driving to the manual driving when one of the acceleration and the three-dimensional tilt is greater than a second angle threshold after the myoelectric potential rises above the reference myoelectric potential; and
switch the autonomous driving to the manual driving after a predetermined time when one of the acceleration and the three-dimensional tilt is equal to or less than the second angle threshold after the myoelectric potential rises above the reference myoelectric potential.

7. The device according to claim 1, wherein the processing circuitry is configured to:
switch the autonomous driving to the manual driving more quickly as the myoelectric potential rises above the reference myoelectric potential at a higher rapidity of change.

8. The device according to claim 1, wherein the processing circuitry is configured to:
switch the autonomous driving to the manual driving when a duration time over which the myoelectric potential remains above the reference myoelectric potential is longer than a time threshold.

9. The device according to claim 3, wherein the processing circuitry is configured to:
acquire the acceleration of both arms of the user; and
switch the autonomous driving to the manual driving when the myoelectric potential is higher than the reference myoelectric potential and relevance of one of the acceleration and three-dimensional tilts between the both arms is greater than a first relevance threshold.

10. The device according to claim 3, wherein the processing circuitry is configured to:
acquire the acceleration of both arms of the user;
switch the autonomous driving to the manual driving when the myoelectric potential is higher than the reference myoelectric potential and relevance of one of the acceleration and three-dimensional tilts of the both arms is greater than a second relevance threshold; and
switch the autonomous driving to the manual driving after a predetermined time when the myoelectric potential is higher than the reference myoelectric potential and relevance of the three-dimensional tilts is equal to or less than the second relevance threshold.

11. An information processing method of a mobile object movable under autonomous driving and manual driving by a user using an information processing device of the mobile object, the method comprising:
acquiring myoelectric potential of at least one part of a body of the user;
determining whether the myoelectric potential is higher than reference myoelectric potential and switching the autonomous driving to the manual driving when the myoelectric potential is higher than the reference myoelectric potential; and
outputting running control information to the mobile object after the autonomous driving is switched to the manual driving.

12. The method according to claim 11, further comprising:
acquiring at least acceleration in time sequence as to motion of the part of the body of the user; and
generating the running control information according to the acceleration after switching the autonomous driving to the manual driving.

13. The method according to claim 12, further comprising:
acquiring a three-dimensional tilt in time sequence found from the acceleration of three axes on a three-dimensional space, wherein
the generating generates the running control information according to the three-dimensional tilt after switching the autonomous driving to the manual driving.

14. The method according to claim 11, wherein
the switching switches the autonomous driving to the manual driving when the myoelectric potential is higher than both of the reference myoelectric potential and a myoelectric potential threshold; and
the switching switches the autonomous driving to the manual driving after a predetermined time when the myoelectric potential is higher than the reference myoelectric potential and equal to or lower than the myoelectric potential threshold.

15. The method according to claim 13, wherein
the switching switches the autonomous driving to the manual driving when one of the acceleration and the three-dimensional tilt becomes greater than a first angle threshold after the myoelectric potential rises above the reference myoelectric potential.

16. The method according to claim 13, wherein
after the myoelectric potential rises above the reference myoelectric potential, the switching switches the autonomous driving to the manual driving when one of the acceleration and the three-dimensional tilt is greater than a second angle threshold; and
after the myoelectric potential rises above the reference myoelectric potential, the switching switches the autonomous driving to the manual driving after a predetermined time when one of the acceleration and the three-dimensional tilt is equal to or less than the second angle threshold.

17. The method according to claim 11, wherein
the switching switches the autonomous driving to the manual driving more quickly as the myoelectric potential rises above the reference myoelectric potential at a higher rapidity of change.

18. The method according to claim 11, wherein
the switching switches the autonomous driving to the manual driving when a duration time over which the myoelectric potential remains above the reference myoelectric potential is longer than a time threshold.

19. The method according to claim 13, wherein
the acquiring acquires the acceleration of both arms of the user; and
the switching switches the autonomous driving to the manual driving when the myoelectric potential is higher than the reference myoelectric potential and relevance of one of the acceleration and three-dimensional tilts between the both arms is greater than a first relevance threshold.

20. A non-transitory program stored in a computer readable medium, the program being an information processing program of a mobile object movable under autonomous driving and manual driving by a user and causing a computer to perform:
an acquisition function of acquiring myoelectric potential of at least one part of a body of the user;
a determination function of determining whether the myoelectric potential is higher than reference myoelectric potential and switching the autonomous driving to the manual driving when the myoelectric potential is higher than the reference myoelectric potential; and
a control function of outputting running control information to the mobile object after the autonomous driving is switched to the manual driving.
